(19) United States
(12) Patent Application Publication
(10) Pub. No.: US 2026/0285778 A1
(43) Pub. Date: Sep. 24, 2026

(54) A Device And A Method For Composting Using Larvae

(71) Applicant: Samabio OY, Perniö As (FI)

(72) Inventor: Santtu Juvonen, Perniö As (FI)

(73) Assignee: Samabio OY, Perniö As (FI)

(21) Appl. No.: 19/470,040

(22) PCT Filed: Mar. 21, 2024
(86) PCT No.: PCT/FI2024/000005

§ 371 Date: Sep. 27, 2025

(30) Foreign Application Priority Data
Mar. 27, 2023 (FI) 20237059

(51) Int. Cl. C05F 17/907; C05F 17/964

(52) U.S. Cl. CPC C05F 17/907; C05F 17/964

(57) ABSTRACT

The present disclosure is directed to a device for composting organic material using larvae. The device comprises a container configured for the organic material and larvae. The container comprises a first end and a second end. The container is positioned in the device such that when the device is in use the first end is arranged and/or located higher than the second end. The container further comprises walls and an interior area. The walls comprise a multitude of holes extending through the walls. The holes are sized such that larvae can exit and return through the holes. Size of the container is such that a heated area is formed in a bed of organic material located in the interior area. A temperature of the heated area is 40 C or more.

### CROSS REFERENCE TO RELATED APPLICATIONS

This application is a national phase application of international application PCT/FI2024/000005, filed 21 Mar. 2024, which claims priority to Finnish patent application FI20237059(A1), filed Mar. 27, 2023, the content of the both of which are herein incorporated by reference.

### BACKGROUND OF THE INVENTION

### Field of the Invention

The invention relates to a device for composting organic material using larvae, and the device comprises a frame, and the frame comprises a container for the organic material and larvae, and the container comprises a first end of the container and the second end of the container, and the container is positioned in the device in such a way that when the device is in use the first end of the container is higher than the second end of the container. The invention relates also to a method for composting using larvae.

### Description of Related Art

It is a well-known fact that biowaste treatment processes, even controlled processes, can create environmental problems such as release of green-house gases (methane, carbon dioxide) and production of gaseous smelly by-products. Leakage of methane to atmosphere can happen also in biogas reactors. Biological treatments are dominated by composting and anaerobic processes for biogas pro-duction. Aside managing biowaste, these processes produce energy and valuable by-products such as frass and energy.

Anaerobic processes are not well-fit for all types of biowaste. Fibre rich wastes like wood, leaves, etc. make poor feedstocks for biogas production, as anaerobic microbes are not capable of degrading lignin. Also too high nitrogen content of the feed can lead to inhibition. After the process, sludge is not yet applicable to agricultural purposes, since during biogas production pathogenic microbes (bacteria, protozoa) are not completely destroyed. For the use as fertilizer, the sludge needs to be further composted. Anaerobic processes are also quite slow—the starting phase can last several weeks. typically biowaste process lasts several months.

Aerobic composting means degradation of biowaste to nutrients in the presence of oxygen. Suitable substrates include yard waste (branches, leaves, grass), food waste, agricultural waste, manure, and even septage and human feces . The resulting mixture is rich in plant nutrients and beneficial organisms, such as bacteria, protozoa, nematodes and fungi. Aerobic conditions enable the growth of oxygen-demanding organisms, which can efficiently degrade woody material. As a result of the work of the microorganisms, water or steam, carbon dioxide, nutrients and thermal energy are released from the waste. At the same time, humus, which is important for the soil's fertility, is formed. Compost micro-organisms use both compostable waste and each other as food. With their metabolism, heat is produced in the compost. Thereby the compost can easily heat up to 70-80 C, which is an advantage for the formation of high-quality compost soil. Composting process can be artificially enhanced by piling biowaste to heaps, confirming that suitable content of carbon, oxygen, nitrogen and water is present. During the composting process, heat-tolerant microbes take care of efficient biomass decaying and at the same time destroy harmful microbes and the seeds of weeds. After the heat-generating step, composts is allowed cool down and stabilized. During this phase, the amount of phytotoxic compounds will decrease, and the compost will be inhabited by organisms (fungi, bacteria) which can further continue biowaste degradation.

Biowaste management with insect is gaining an increased amount of attention. Insect can both enhance and accelerate biowaste conversion to frass and con-vert biowaste to their biomass, which provides valuable ingredient for food and animal feed: particularly insect protein and oil. Especially insect larvae are found to be very efficient. This method is called vermicomposting, and in that decomposition process uses various species of worms or insect larvae to create a mixture of decomposing vegetable or food waste, bedding materials, and vermicast. As the larvae feed on the waste, they will break down the organic matter and metabolize the nutrients into larval biomass. Vermicast, the end-product of the breakdown of organic matter, have been shown to contain reduced levels of contaminants and a higher saturation of nutrients than the organic materials before vermicomposting. Vermicomposting can also be applied for treatment of sewage.

There are two main methods of large-scale vermicomposting, windrow and raised bed. Some systems use a windrow, which consists of bedding materials for the earthworms to live in and acts as a large bin, organic material is added to it. Although the windrow has no physical barriers to prevent worms from escaping, in theory they should not, due to an abundance of organic matter for them to feed on. Often windrows are used on a concrete surface to prevent predators from gaining access to the worm population. The second system is the raised bed or flow-through system, which operates on continuous basis. Here the worms are fed across the top of the bed, and the castings are harvested from below by pulling a breaker bar across the large mesh screen which forms the base of the bed. Because worms are constantly moving towards the new food source, the flow-through system eliminates the need to separate worms/larvae from the castings before packaging. Flow-through systems are well suited to indoor facilities, making them the preferred choice for operations in colder climates or indoor composting. Small-scale vermicomposting is well-suited to turn kitchen waste into high-quality soil amendments, where space is limited. Worms can decompose organic matter without the additional human physical effort (turning the bin) that bin composting requires.

Large scale vermicomposting with insect larvae, based on windrow or raised bed principles meets several challenges. Insect larvae at the late development status (prepupae) tend to leave the moist biomass and search for suitable dry place for pupation and development of to (flying) adults. This requires that escape of larvae is prevented and harvesting (removal) or killing of larvae is enabled prior to the use of the processed biomass (frass). Such open systems also make the biomass prone to the invasion by competing insects (such as houseflies and fruit flies), the access of which is difficult to be prevented. Cold climate sets limitations to vermicomposting. Since thermoresistant heat-generating microbes are not involved, vermicomposting does not generate heat. Therefore, its use is limited to warm (but not too hot) conditions.

Characteristics of the known vermicomposting systems is that the operating temperature should be moderate, generally below 35 C. These systems actually may have reduced usability in tropics, since the favoured temperature ranges of worms is 16 to 25 C. For this reason, the hygienization of biomass will be incomplete. Generally, it is agreed that vermicomposting reduces the amount of harmful bacteria, however, since it provenly cannot kill all pathogens, frass needs to be hygienized with other means after the treatment.

Rearing insect larvae for food and feed conventionally applies vertical farming using stacked boxes or crates. Use of boxes or crates with low value and/or coarse biowaste is laborious, time-consuming and unpractical. It should be also noted that especially lignocellulosic plant-based feed does not provide much energy and nutrients for economically feasible cultivation of larvae for protein production. Consequently, box or tray based cultivation systems cannot be regarded as suitable cultivation system for converting coarse woody low-value material to larvae for the use for insect protein production.

Manure-treatment brings extra challenges. Due to the presence of pathogenic microbes, manure is generally not regarded as approved material for insect protein production. For these applications, frass is regarded as the primary product and insect larvae as a secondary product. The dry matter content of larvae can have up to 30% of oils, and this oil can be applied to biodiesel production. Large scale processes in windows or raised compost beds bring challenges in harvesting. Therefore, the potential for biodiesel production from low-value biowaste and manure by vermicomposting remains unused.

Thermal inactivation may be requested, before the frass is accepted as fertilizer. Especially manure can contain a high content of pathogenic organisms, which are not destroyed during normal vermicomposting or biogas production processes. Conventional vermicomposting process does not include hygienization of the frass. Hygienization of frass may therefore need (depending on the local regulations) either heat-treatment (>70 C) or freezing. As we will present in this patent application, with the presented instrumentation it is possible to raise the temperature by intrinsic heat generation to 70 C for several hours, and thus perform hygienization for the vermicompost frass.

One example of insects that fit well for vermicomposting are black soldier fly larvae (Hermetia illucens). They are naturally waste scavengers and grow in moist conditions feeding on decaying waste. They may significantly enhance the composting process, as their intestine provide specialized mini-environments suitable for various different microbes. Many studies have showed that black soldier fly larvae strengthen the metabolic function of food waste biodegradation by gut microbiome, making gut microbiome an engine for biowaste conversion.

However, there is general opinion that if too much organic material to be composted is used, an unprocessed layer bed will build up heat through bacterial activity and, thus, make the environment unfavourable and even lethal for the larvae. Also, there is an opinion that if the depth of the waste bed is more than 5 cm, larvae will have difficulties to process it entirely. Patent publication U.S. Pat. No. 10,842,138 shares these views. It describes a method and a device for cultivating insect larvae, processing organic material and harvesting of larvae and frass. It discloses a larvae tray that is a curved, shallow structure which rests on the shelf support rails, and the total depth of the tray from the bottom of the curve to the top of the upper support piece is approximately 6 inches (15 cm). Also, the publication states that larvae colony is maintained in a controlled environment at a temperature of 75-95 degrees Fahrenheit (24-35 C).

### BRIEF SUMMARY OF THE INVENTION

An object of the invention is a solution that can significantly reduce the dis-advantages and drawbacks of the prior art. In particular, an object of the invention is a solution where a device is provided that allows efficient composting process using larvae.

The objects of the invention are attained with an arrangement that is characterised by what is stated in the independent patent claims. Some advantageous embodiments of the invention are disclosed in the dependent claims.

The invention is a device for composting organic material using larvae. The device comprises a container for the organic material and larvae. The container comprises a first end of the container and the second end of the container, and the container is positioned in the device in such a way that when the device is in use the first end of the container is higher than the second end of the container. The container further comprises walls and an interior area of the container, and the walls comprise a multitude of holes extending through the walls. Size of the holes is such that larvae can and return through the holes. Size of the container is such that a heated area is formed in bed of the organic material in the interior area, and temperature of the heated area is 40 C or more.

When reference is made in the text to the upper or the lower parts or respective directions such as down or up, a situation is described in which the device according to the invention is resting on a surface such as a floor. Also, when reference is made to vertical or horizontal directions or surfaces, the device is placed similarly. The direction of the gravity defines the vertical direction.

In one embodiment of the invention is a device for composting organic material using larvae, and the device comprises a container for the organic material and larvae, and the container comprises a first end of the container and the second end of the container, and the container is positioned in the device in such a way that when the device is in use the first end of the container is higher than the second end of the container. The container further comprises walls of the container and an interior area of the container. The walls of the container surround the interior area of the container. In one advantageous embodiment of the invention, the walls of the container surround the interior area of the container, and the walls comprise a multitude of holes extending through the walls, and diameter of the holes is such that it allows larvae exit from the interior area and return to the interior area through the holes. Dimensions of the interior area of the container are such that it allows a bed of the organic material to be added to the interior area of the container where a composting process forms a heated area in centre area of the bed of the organic material, and temperature of the heated area is 40 C or more. Diameter of the interior area of the container is 40 cm or more, and heights of the walls of the container are such that they allow vertical thickness of the bed of the organic material 40 cm or more. The device further comprises a collar structure surrounding the container at least partly, and the collar structure comprises walls of the collar structure, and there is a gathering area between the walls of the collar structure and the walls of the container, and the collar structure is positioned in such a way that the larvae exiting from the interior area through the holes end up on the gathering area.

In one embodiment of the device, the larvae are black soldier fly larvae.

In a second embodiment of the device, the minimum diameter of the holes in the walls of the container is 5 mm.

In a third embodiment of the device, the walls of the container are vertical or deviate from the vertical direction 5 degrees at maximum, when the device is in use.

In a fourth embodiment of the device, some of the holes in the walls of the container are positioned in such a way that the larvae are able to enter to the interior of the container from the gathering area.

In a fifth embodiment of the device, the walls of the collar structure comprise an edge of the walls of the collar structure, and on the edge of the walls of the collar structure is a guidance arrangement for preventing the larvae crossing the walls of the collar structure.

In a sixth embodiment of the device, the device further comprises one or more additional collar structure positioned between the collar structure and the first end of the container, and the additional collar structure is configured to form a gathering area of the additional collar structure between the additional collar structure and the wall of the container.

In a seventh embodiment of the device, the device further comprises a sieve arrangement at the second end of the container, and the sieve arrangement is configured to allow liquids from the interior area drain through the sieve arrangement. In an eighth embodiment of the device, the device further comprises a collector arrangement for collecting liquids that have gone through the sieve arrangement.

In a nineth embodiment of the device, the device further comprises a lower area of the device, and the lower area of the device is configured to be closest to a surface on what the device or a support structure of the device is positioned when the device is in use, and the lower area of the device and the first end of the container are shaped in such a way that one device is stackable upon another device, i.e. the lower area of one device is configured to fit over the first end of the container of another device, and the sieve arrangement of one device is configured to allow liquids drain from one device to another device, i.e. from an upper device to a lower device. In a thirteenth embodiment of the device, the sieve arrangements of the consecutive devices are configured in such a way that the sieve arrangement on the lower device allow more liquid going through it than the sieve arrangement on the upper device, i.e. the sieve arrangement of the lower device is more liquid permeable than the sieve arrangement of the upper device.

One embodiment of the invention is a method for composting organic material using larvae. The container is for the organic material and larvae, and the container comprises a first end of the container, a second end of the container, walls of the container and an interior area of the container, and the container is positioned in the device in such a way that when the device is in use the first end of the container is higher than the second end of the container, and the walls of the container surround the interior area of the container. The walls comprise a multitude of holes extending through the walls of the container, and diameter of the holes is such that it allows larvae exit from the interior area and return to the interior area through the holes, and dimensions of the interior area of the container are such that it allows a bed of the organic material to be added to the interior area of the container where a composting process forms a heated area in centre area of the bed of the organic material, and temperature of the heated area is 40 C or more. The method comprises steps where organic material is added to the interior area until a bed of the organic material is formed, and volume of the bed is sufficient to sustain a heated area forming by a composting process, the larvae are added to the bed of the organic material, and both the larvae and the composting processes decompose the organic material, and the device is configured to allow the larvae exit the interior of the container and/or provide areas near the walls of the container where temperature is suitable for the larvae. When the organic material is sufficiently composted, decomposed organic material and the larvae are removed and separated.

In one embodiment of the method, the method comprises a step where the composted organic material is removed.

In a second embodiment of the method, the method comprises a step where temperature of the interior area of the container is increased by accelerating the composting processes, and the rising temperature forces the larvae migrate away from the interior area.

In a third embodiment of the method, the device comprises a collar structure surrounding the container at least partly, and the collar structure comprises walls of the collar structure, and there is a gathering area between the walls of the collar structure and the walls of the container, and the method comprises a step where the migrating larvae end up to the gathering area where they are collected.

In a fourth embodiment of the method, the method comprises a step where a new patch of organic material added to the device after the larvae are removed.

In a fifth embodiment of the method, the method comprises a step where new organic material added to the device at regular or semi-regular phases.

In a sixth embodiment of the method, the method comprises a step where a new batch of larvae is added to the device after harvesting the previous batch of larvae.

It is an advantage of the invention that it provides a device that improves composting processes significantly. Also, the invention decreases the possibility of unwanted smells.

The invention is also suitable for many kinds of organic material. Even low value biowaste and biowaste with high content of lignocellulosic materials can ap-plied. Also, the organic material can be left coarse. Using the invention makes process faster and more efficient and allows alternative conventional processes for biowaste treatment.

One advantage of the invention is that the construction of the device is easy. The operating costs of the invention are significantly less than the operating costs of the known devices. The invention allows a system that can be easily scaled up either by building larger devices or by building more of them. Furthermore, the device according to the invention can be operated either in batch mode or as a continuous process. In contrast conventional larvae cultivation processes are typically batch processes (when performed in boxes or trays) or fed-batch process (i.e. insect bioreactors). Swapping between appropriate cultivation mode does not require changes to the structure of the device according to the invention.

It is a further advantage of the invention that it prevents larvae spreading outside of the device and harvesting larvae is easy and does not require special instrumentation.

Also, it is an advantage that composting process, especially the heating phase, is enhanced and accelerated by the action of larvae, which mix biomass and produce cavities which enhance the gas exchange. Therefore, the frass (un-eaten biomass) is more mature and more applicable a fertilizer.

Because the process generates heat, frozen biomass or organic material can be fed to the process. Also, especially when black soldier fly larvae are used, high operating temperature kills competing insects, which cannot tolerate temperatures close to 50 C. Furthermore, black soldier fly larvae produce antimicrobial substances (e.g. lauric acid) and antimicrobial peptides which can suppress the growth of pathogenic microbes or even kill them. Also, the inventor has found that using higher temperatures than are used in the conventional vermicomposting processes, significantly reduces amount of smells the larvae produces.

### BRIEF DESCRIPTION OF THE SEVERAL VIEWS OF THE DRAWINGS

Further advantageous features and details of the various embodiments of this disclosure will become apparent from the ensuring description of a preferred exemplary embodiment or embodiments and further with the aid of the drawings. The features and combinations of features recited below in the description, as well as the features and feature combination shown after that in the drawing description or in the drawings alone, may be used not only in the particular combination recited by also in other combinations on their own without departing from the scope of the disclosure.

In the following, the invention is described in detail. The description refers to the accompanying drawings, in which:

FIG. 1 shows a simplified example of a device in accordance with an embodiment,

FIG. 2 shows a second example of a device in accordance with an embodiment, and

FIG. 3 shows the device of FIG. 2 after larvae has been added.

### DETAILED DESCRIPTION OF THE INVENTION

As used throughout the present disclosure, unless specifically stated otherwise, the term “or” encompasses all possible combinations, except where infeasible. For example, the expression “A or B” shall mean A alone, B alone, or A and B together. If it is stated that a component includes “A, B, or C”, then, unless specifically stated otherwise or infeasible, the component may include A, or B, or C, or A and B, or A and C, or B and C, or A and B and C. Expressions such as “at least one of” do not necessarily modify an entirety of the following list and do not necessarily modify each member of the list, such that “at least one of “A, B, and C”.

The embodiments in the following description are given as examples only. Though the description may refer to a certain embodiment or embodiments in several places, this does not mean that the reference would be directed towards only one described embodiment or that the described characteristic would be usable only in one described embodiment. The individual characteristics of two or more embodiments may be combined and new embodiments of the invention may thus be provided.

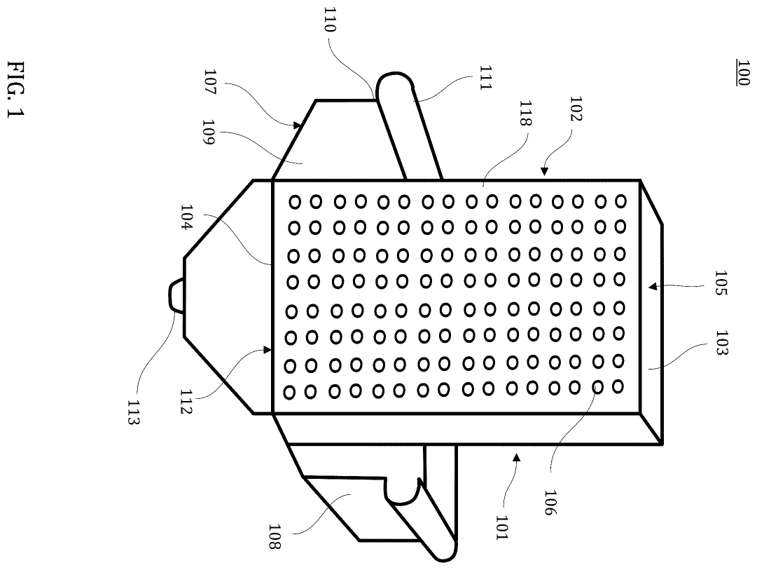

FIG. 1 shows a simplified example of an embodiment of a device 100. The device is for composting organic material using larvae. The device comprises a frame 101. The frame comprises a container 102 for the organic material and larvae. In some embodiments, the frame further comprises a support structure. The support structure is configured to support the container and keep the device in a stable position during the use of the device. In FIG. 1 the device is partly cut open for explaining the invention.

The container 102 comprises a first end 103 of the container and a second end 104 of the container, and the container is positioned in the device 100 in such a way that when the device is in use, the first end of the container is higher than the second end of the container. This means that when the container is filled with the organic material gravity pulls the organic material downwards from the first end of the container towards the second end of the container.

The container further comprises walls 118 of the container and an interior area 105 of the container. The walls of the container surround the interior area of the container. In this embodiment, the walls 118 of the container are vertical or deviate from the vertical direction 5 degrees at maximum, when the device 100 is in use. The walls comprise a multitude of holes 106 extending through the walls of the container (all holes are not depicted due clarity of Figure). Diameter of the holes is such that it allows larvae exit from the interior area and return to the interior area through the holes. This means that minimum diameter of the holes is bigger than maximum diameter of the larvae have for being capable entering through the hole. Because larvae are elongated organisms this diameter is not the maximum length of the larvae. It must be noted that the walls 118 of the container may comprise holes that are smaller than the larvae. These kinds of holes are for additional ventilation. In the embodiment of FIG. 1 the holes are circle shaped. Naturally, other shapes can be used. For example, the shape of the holes can square or rectangular. In some embodiments, shapes and sizes of the holes vary. For example, in some embodiments, the diameters of the holes are bigger near the second end of the container than near the first end of the container, i.e. the holes are bigger in bottom parts of the device. In some embodiments, the walls of the container are formed using net or grid comprising bars or similar structures. Empty space between bars are the holes.

The first end 103 of the container is open, i.e. the container comprises an opening at top of the container, and the organic material is fed to the interior area 105 through the opening. The organic material forms a bed of the organic material in the interior area. The dimensions of the interior area of the container are such that a composting process or processes generate a heated area in centre area of the bed of the organic material. Temperature of the heated area is 40 C or more. This ensures that degradation of the organic material in composting processes is faster and more complete than with conventional vermicomposting methods. Compost heat is produced as a by-product of the microbial breakdown of the organic material. The heat production depends on the size of the bed, its moisture content, aeration, and properties of the organic material. The inventor has found out that minimum diameter of to the interior area of the container is 40 cm or more, and heights of the walls of the container are such that they allow vertical thickness of the bed of the organic material 40 cm or more (i.e. the walls of the container should be 40 cm or more). When the organic material is piled in a container having that kind of dimensions a heated area can be formed in the bed of the organic material. The process temperature can be controlled by controlling ventilation or preventing the escape of heat by covering the device or controlling moisture of the bed of the organic material or some combination of these methods.

The inventor has found out, on contrary to common opinion, that larvae can survive and feed in a compost material that comprise areas having temperatures over 40 C or even over 50 C. This is due to the structure of the device 100 that allows the larvae exit the interior area 105 of the container 102 through the holes 106 of the walls 118 of the container to cooler areas and enter back into the container. Also, the holes provide cooler areas in the bed of organic material where the temperatures of the cooler areas are less than the temperatures of the heated areas. The device provides a system where larvae are allowed to migrate from heated areas to cooler areas. One especially good candidate for using the invention is black soldier fly and its larvae. Inventor has found that the black soldier fly larvae are particular resistant to temperatures that have previously been thought as lethal for the larvae. This is possible when the black soldier fly larvae are provided a possibility to visit cooler areas. Also, the black soldier fly larvae strengthen the metabolic function of food waste bio-degradation by gut microbiome, making gut microbiome an engine for biowaste conversion. When the cooler areas are provided, the microbes attained in the gut of larvae are protected from overheating. Consequently, the black soldier fly larvae enable the maintenance of a very wide variety of microbial enzymatic activities (with different temperature optima) and allow (at least temporary) enhancement of enzymatic activities when the black soldier fly larvae migrated to the heated nutrient-rich areas of the compost.

Insect larvae, particularly the black soldier fly larvae, mix efficiently the bed of the organic material (i.e. a compost). This improves ventilation and promotes the activity of microbes which in composting efficiently degrade the organic material. Due to their action, the heat-generating phase in composting process starts earlier and is enhanced. Therefore cold, even frozen, biomass can be applied to the process. The black soldier fly larvae particularly have physiology and metabolism ideal to biowaste degradation. The device 100 can be operated at temperatures which are at level or even above the lethal temperature of the black soldier fly larvae. This prevents the growth of competing insect species (like house flies, fruit flies, flower flies) which feed on decaying plant material.

Elevated temperatures enhance the activity of several enzymes. Generally, increase of 10 C enhances the activity 2 to 4 fold. Several thermostable enzymes capable of degrading them at temperatures of 50 C or higher have been identified, especially in fungi and bacilli. Biowaste decaying can therefore be enhanced by temperature increase. This probably makes many slowly decaying components better available for the black soldier fly larvae (or other larvae), particularly lignin and cellulose. As a consequence, efficient composting process (biowaste decaying at high temperature using aerobic thermophilic organisms) can proceed during the black soldier fly larvae rearing. The black soldier fly larvae also efficiently mix the biowaste feed and create cavities which enhance ventilation and improve the growth conditions for thermophilic aerobic organisms. The device 100 according to the invention can therefore efficiently combine the benefits of composting and vermicomposting.

In this embodiment, the second end 104 of the container 102 is open. The device 100 further comprises a sieve arrangement 112 at the second end of the container or near it. The sieve arrangement forms a bottom for the interior area 105 of the container. The bed of the organic material rests on the sieve arrangement. This means that the walls of the container and the sieve arrangement define the interior area. The sieve arrangement comprises a multitude of a sieve holes. The sieve arrangement is configured to allow liquids from the interior area drain through the sieve arrangement. In some embodiments, the device comprises a collector arrangement 113 under the sieve arrangement. The collector arrangement is for collecting liquids that have gone through the sieve arrangement. In some embodiments, the collector arrangement is formed under the device. That kind of collector arrangement may be, for example, a recess on a surface where the device is positioned, and there is a liquid gathering system in the recess. The liquid that is generated by the composting processes and goes through the sieve arrangement is called compost tea. The collected compost tea is usually circulated back to the system.

In this embodiment, the device 100 further comprises a collar structure 107 surrounding the container 102 at least partly. The collar structure comprises walls 108 of the collar structure and an edge 110 of the walls of the collar structure. On the edge of the walls of the collar structure is a guidance arrangement 111 for preventing the larvae crossing the walls of the collar structure. There is a gathering area 109 between the walls of the collar structure and the walls 118 of the container. The collar structure is positioned and shaped in such a way that the larvae exiting from the interior area 105 through the holes 106 end up on the gathering area. Some of the holes 106 in the walls of the container are positioned in such a way that the larvae are able to enter back to the interior of the container from the gathering area. The collar structure is positioned in such a way that the lowest holes (the closest to the second end of the container) are open to the gathering area (i.e. it is not possible for larvae to enter below the interior area from the gathering area). The gathering area works for a cooling area for larvae. Also, by adjusting process conditions (temperature) in the interior area of the container, the larvae can be made to leave the feeding area (the interior area) and to accumulate to the gathering area from which they can be easily collected.

In this embodiment, the horizontal cross section of the container 102 is rectangular. Naturally, it can be any shape as long as the shape of the container provides the interior area where the bed of the organic material, that is big enough for generating the heated area, can be formed.

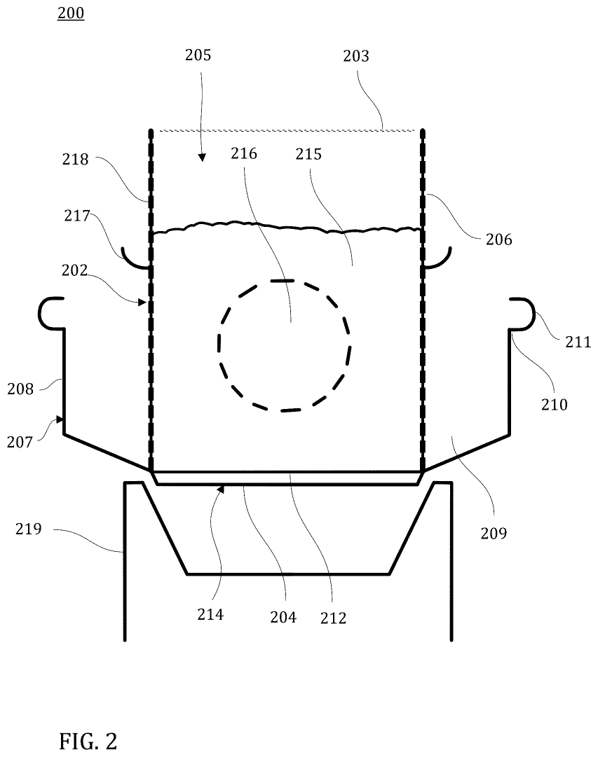

FIG. 2 shows a second example of an embodiment of a device 200 for composting organic material using larvae. The device comprises a container 202, a first end 203 of the container, a second end 204 of the container, walls 218 of the container, an interior area 205 of the container, a multitude of holes 206 extending through the walls of the container, a sieve arrangement 212, and a collar structure 207. In the interior area is a bed 215 of organic material and in centre area of the bed is a heated area 216. The device further comprises a lower area 214 of the device, i.e. a bottom area. In this example, the device is resting on a support structure 219. In some embodiments, the support structure is a part of the device and in some of said embodiments, the support structure comprises the collector arrangement.

In this embodiment, the device 200 further comprises an additional collar structure 217. The collar structure comprises walls 208 of the collar structure and an edge 210 of the walls of the collar structure, and on the edge of the walls of the collar structure is a guidance arrangement 211. Between the walls of the collar structure and the walls 218 of the container is a gathering area. The additional collar structure is positioned between the collar structure and the first end of the container. The additional collar structure is configured to form a gathering area of the additional collar structure between the additional collar structure and the wall of the container. In some embodiments, the device comprises more than one additional collar structure.

The lower area 214 of the device 100 and the first end 203 of the container 202 are shaped in such a way that one device is stackable upon another device, i.e. the lower area of one device is configured to fit over the first end of the container 202 of another device. In this embodiment, the lower area is shaped in such a way that its surface are slightly inclined inwards. This inclined part of the device can be fit inside of another device's container. Of course, this piling of two or more devices can be implemented in many ways. The sieve arrangement 212 of one device is configured to allow liquids drain from one device to another device, i.e. from an upper device to a lower device. In some embodiments, the sieve arrangements of the consecutive devices are configured in such a way that the sieve arrangement on the lower device allow more liquid going through it than the sieve arrangement on the upper device, i.e. the sieve arrangement of the lower device is more liquid permeable than the sieve arrangement of the upper device. The lowest device in the pile of devices comprise the support structure in some embodiments.

The organic material is added through an opening at the first end 203 of the container. In some embodiments, the opening is covered by a lid that is configured to be removed or opened when the interior area 205 is to be reached, for example for measuring compost process conditions or for adding organic material or process enhancing material in the bed 215. In some embodiments, the first end of the container is open.

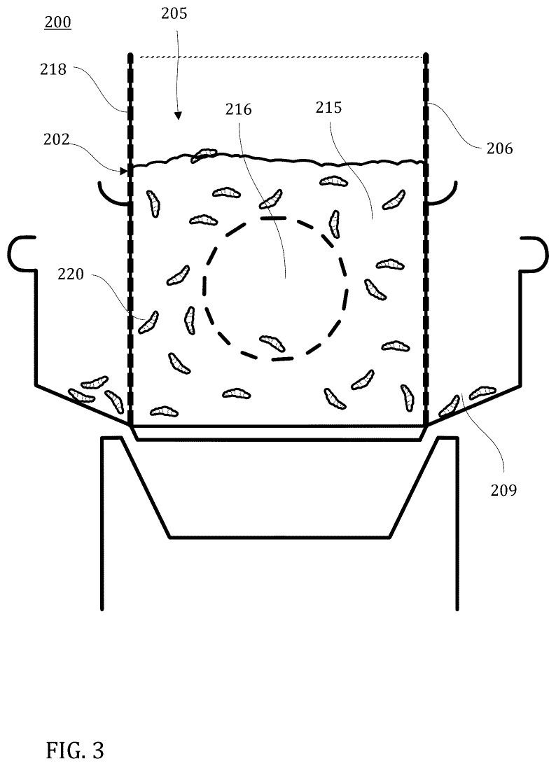

With FIG. 3 some examples of the method using the device 200 is described. Even if the method is described using embodiment of device presented in FIG. 2, the method is applicable with other embodiments of the device.

Organic material is added into the interior area 205 of the container 202 and the bed 215 of the organic material is formed. Larvae 220 are added to the bed. Both microbial and larvae activity start produce heat in middle of the bed and thus the heated area 216 is formed. The larvae migrate cooler areas near the walls 218 of the container or crawl out of the container 202 through the holes 206. When the larvae have cooled, they return to the heated area or near it, because composting process produces nutrients the larvae can feed. If the larvae end up on the gathering area 209, they can enter back the interior area through the holes near the gathering area.

Composting processes happens in phases, wherein different microorganisms prevail provided that nutrients, pH, oxygen availability and temperature are favourable. Therefore, degradation of different organic compounds does not proceed in parallel. Especially efficient degradation of fibres and woody material occurs primarily the late phase (stabilization phase) of composting. Added insect larvae significantly enhance the composting process by 1) enhancing maceration of biowaste to smaller particles 2) moving biowaste inside compost 3) since larvae intestine has both aerobic and anaerobic sections, they provide favourable conditions for both aerobic and anaerobic microbes and thus improve the diversity of microbial activities, and 4) they spread around and transfer microbes to various temperature clines inside the compost. This is important, since some enzymes digesting cellulose, hemicellulose or lignin can be active in fairly high temperatures, although the microorganism them-selves may not survive in such conditions. Due these enhancements many kinds of low-value organic material and organic material with high content of lignocellulosic materials can applied for the composting. These are especially evident when using black soldier fly larvae, but in some embodiments larvae of other insect species may be suitable.

Inventor has found that biomass degradation using the invention is faster and more complete compared to conventional black soldier fly larvae cultivation processes, since the process occurs at high temperature (45 to 55 C), whilst conventional black soldier fly rearing usually applies 25-30 C, and vermicomposting with worms 16-25 C.

In some embodiments, prior adding larvae, organic material can be pre-processed by fermenting it with organisms that improve its digestibility for larvae and neutralize the smell of the decaying biomass. Alternatively, liquid extract from previously fermented biomass can be added to neutralize unwanted odour. This pre-fermenting can be performed in the same container.

The device 200 can be operated either in a batch mode or as a continuous process. In the batch mode, the bed 215 is formed in the container 202 and the organic material is composted. In the continuous process, organic material is added at regular (or semi-regular) phases.

When the composting process is ready, or when the larvae 220 are to be harvested, the temperature on the composted material is raised so much that the larvae migrate out from the container 202. If the device comprises the gathering area 209, the larvae can be easily collected from there. The raising of temperature is achieved by adjusting some of the process conditions. When the larvae have been harvested, a new batch of larvae can be added, or the composted organic material is collected.

Finally, it should be noted that the description of the invention and the exemplary embodiments are not to be understood as limiting in terms of a particular physical realisation of the invention. The scope of protection of the present invention is given by the claims and is not limited by the features illustrated in the description or shown in the figures.

1. A device for composting organic material using larvae, the device comprising:
a container configured to accommodate the organic material and the larvae, the container comprising a first end and a second end, the container arranged in the device such that when the device is in use the first end is located above or higher than the second end, and the container further comprising walls and an interior area, wherein the walls surround the interior area;
wherein the walls comprise a multitude of holes extending through the walls, the boles comprising a diameter configured to enable larvae to exit from the interior area and return to the interior area through the holes, and
wherein the interior area is dimensioned to enable addition of a bed of organic material to the interior area where a composting process forms a heated area in a center area of the bed of organic material, such that temperature of a heated area is 40 C or more,
wherein the interior area comprises a diameter of 40 cm or more,
wherein heights of the walls are configured such that they allow vertical thickness of the bed of the organic material 40 cm or more,
wherein the device further comprises a collar structure arranged to at least partially surround the container, the collar structure comprising walls of the collar structure arranged so as to define a gathering area between the walls of the collar structure and the walls of the container, and the collar structure is positioned such that the larvae exiting from the interior area through the holes end up on the gathering area.

2. The device according to claim 1, wherein the larvae are black soldier fly larvae.

3. The device according to claim 1, wherein a minimum diameter of the holes in the walls of the container is 5 mm.

4. The device according to claim 1, wherein the walls of the container are vertical or deviate from a vertical direction by 5 degrees at a maximum, when the device is in use.

5. The device according to claim 1, wherein at least some of the holes in the walls of the container are positioned such that the larvae are able to enter the interior area of the container from the gathering area.

6. The device (100; 200) according to claim 1, wherein the walls of the collar structure further comprise an edge of the walls of the collar structure, and on the edge of the walls of the collar structure is a guidance arrangement configured to prevent the larvae from crossing the walls of the collar structure.

7. The device according to claim 1, wherein the device further comprises one or more additional collar structures arranged on the wall of the container between the collar structure and the first end of the container, and the one or more additional collar structures is configured to form a gathering area of the additional collar structure between the additional collar structure and the wall of the container.

8. The device according to claim 1, wherein the device further comprises a sieve arrangement at the second end, and the sieve arrangement is configured to enable liquids from the interior area to drain through the sieve arrangement.

9. The device according to claim 8, wherein the device further comprises a collector arrangement configured to collect liquids that have passed through the sieve arrangement.

10. The device according to claim 8, wherein the device further comprises a lower area configured and arranged to be closest to a surface on what the device or a support structure of the device is positioned when the device is in use, wherein the lower area and the first end are shaped such that one device is stackable upon another device such that the lower area of one device is configured to fit over a first end of a container of another device, and the sieve arrangement of one device is configured to enable liquids to drain from one upper device to another lower device.

11. The device according to claim 10, wherein sieve arrangements of the consecutive devices are configured such that a sieve arrangement on the lower device is configured arranged to enable more liquid to pass therethrough than a sieve arrangement on the upper device such that, the sieve arrangement of the lower device is more liquid permeable than the sieve arrangement of the upper device.

12. A method for composting organic material using larvae, the method comprising the steps of:
arranging a container in a device, the container configured to accommodate the organic material and the larvae, wherein the container comprises a first end, a second end, walls and an interior area,
positioning the container in the device such that when the device is in use the first end is located higher than the second end and the walls surround the interior area,
forming a multitude of holes in the walls, the multitude of holes extending through the walls of the container, and comprising a diameter configured to enable the larvae to exit from the interior area and return to the interior area through the holes,
configuring dimensions of the interior area to enable addition of a bed of the organic material;
adding the bed of organic material to the interior area so as to enable a composting process that forms a heated area in a centre area of the bed such that a temperature of the heated area is 40 C or more;
adding the organic material to the interior area until a bed of the organic material is formed and a volume of the bed is sufficient to sustain the heated area formed by a composting process;
adding the larvae to the bed of the organic material, wherein
both the larvae and the composting processes decompose the organic material, and wherein the device is further configured to enable the larvae to exit the interior area and/or provide areas near the walls of the container where temperature is suitable for the larvae, and
removing the larve when the organic material is sufficiently composted or the larvae are ready for harvesting.

13. The method according to claim 12, further comprising the step of removing the composted organic material.

14. The method according to claim 12, further comprising the steps of increasing the temperature of the interior area of the container by accelerating the composting processes such that the rising temperature forces the larvae migrate away from the interior area.

15. The method according to claim 14, wherein the device further comprises a collar structure at least partly surrounding the container, the collar structure comprising walls configured and arranged to define a gathering area between the walls of the collar structure and the walls of the container, and wherein the migrating larvae end up to the gathering area where they may be collected.

16. The method according to claim 12, further comprising the step of adding a new patch of organic material to the device after the larvae are removed.

17. The method according to claim 12, further comprising the step of adding new organic material to the device at regular or semi-regular phases of intervals.

18. The method according to claim 12, further comprising the step of adding a new batch of larvae to the device after harvesting a previous batch of larvae.
